(19) United States
(12) Patent Application Publication
(10) Pub. No.: US 2007/0173924 A1
(43) Pub. Date: Jul. 26, 2007

(54) Axially-elongating stent and method of deployment

(76) Inventors: Daniel Gelbart, Vancouver (CA); Samuel Victor Lichtenstein, Vancouver (CA)

Correspondence Address: DAN GELBART, 4706 DRUMMOND DR., VANCOUVER, BC, V6T-1B4, CA

(21) Appl. No.: 11/336,803

(22) Filed: Jan. 23, 2006

(51) Int. Cl. A61F 2/06

(52) U.S. Cl. 623/1.15

(57) ABSTRACT

A stent is capable of expanding both radially and axially during deployment. The stent can be balloon expandable or self expanding. In the balloon expandable version, circumferentially expandable structures are connected with folded links. When stent pulled axially, those links allow axial expansion without reducing radial expansion. A specially designed balloon, also capable of radial and axial expansion, is used to deploy the stent. A self-expanding version of this stent can be deployed without a balloon.

### FIELD OF THE INVENTION

The invention is in the field of stents used in the human body.

### BACKGROUND OF THE INVENTION

It has been known that stents are an excellent way to maintain support and prevent blockages of vessels in the human body, particularly blood vessels. They are typically inserted by using a catheter, mostly in conjunction with a balloon angioplasty procedure. Generally there are two types of stents: balloon-expandable (BE) and self-expandable (SE). The BE stents are mounted on a balloon at the end of a catheter. When the balloon is at the correct position, it is expanded by hydraulic pressure, expanding the stent with it. Deflating the balloon allows easy withdrawal from the expanded stent. SE stents are deployed in a similar manner. However, the expansion is powered by elastic forces, most commonly by using “super-elastic” alloys such as Nitinol. Nitinol posses a “shape memory” property, allowing the stent to open up to a previously determined shape by applying mild heat such as body heat. Since most stents are based on a lattice structure emulating continuous material, they tend to get shorter when expanded (as any tube made of continuous material will, because of a property known as Poisson's ratio). There are some designs known as “non shortening stents” which have special features to minimize the shortening. A normal stent will actually shrink when stretched axially, a property used in removable stents. Some SE stents are flexible enough to stretch with the vessel they are in, but in general stents are installed in the final length and either shorten or keep their length during installation.

It is desirable to have a stent that would elongate significantly during installation since it can be navigated through the body while short, making it easy to move around bends in vessels. In minimally invasive surgery the insertion point of the stent can be quite far from the deployment point. Cardiac stents, for example, are inserted via the leg. A short length is an advantage for easier navigation in the body. In some cases, such as leg arteries, a very long stent is needed. Currently this is handled by insertion of multiple stents, as a very long stent will not be sufficiently compact and flexible to be manipulated via the artery. A stent capable of significant axial expansion (on top of the mandatory radial expansion) is of significant benefit.

### SUMMARY OF THE INVENTION

The invention comprises of a special stent design, capable of significant expansion in both the radial and axial direction. A special method and apparatus are needed to deploy such a stent, and they are disclosed as well. In the case of BE stents, the stent comprises of a lattice structure similar to prior art stents for expanding in the radial direction, but also has a secondary structure of folded links allowing it to expand in the axial direction, when stretched, without loss of the radial expansion. The balloon catheter for deploying such a stent has a similar construction: the elastomer the balloon is made of is capable of significant expansion in both directions. The balloon also has some internal reinforcement preventing excessive expansion, as higher pressures then those used for regular stents are required. In the case of an SE stent according to the invention, it can be deployed by a balloon in the conventional manner but can also be deployed with a special tool without the use of a balloon. The tool contains the coiled up SE stent, fully compressed axially. As the stent is pushed out of the tool, it expands both radially and axially. This allows deployment of very long stents without having a long rigid part in the tool.

These and other objects of the invention will become obvious to a person skilled in the art of stents upon reading the description of the invention in conjunction with the drawings.

### BRIEF DESCRIPTION OF THE DRAWINGS

FIG. 1-A is a perspective view of a typical prior art stent before deployment.

FIG. 1-B is a perspective view of the stent shown in FIG. 1-A after deployment.

FIG. 2-A is a perspective view of a stent according to the invention before deployment.

FIG. 2-B is a perspective view of a the stent shown in FIG. 2-A after deployment.

FIG. 3 is an enlarged view of the structure of the stent in FIG. 2-A.

FIG. 4 is an enlarged view of the structure of the stent in FIG. 2-B.

FIG. 5-A is a perspective and cut-away view of the balloon used to deploy the stent, before expanding the balloon.

FIG. 5-B is a perspective and cut-away view of the balloon used to deploy the stent, after expanding the balloon.

FIG. 6 is a perspective view (with a partial cut-away) of the tool used to deploy the self-expanding stent as well as a perspective view of the expanded stent.

FIG. 7 is a cross section of the self expanding stent being deployed inside a vessel.

FIG. 8 is a cross section of a long self-expanding stent being deployed inside a curved vessel.

FIG. 9 is a cross section of a high expansion ratio balloon in the deflated state.

### DETAILED DESCRIPTION OF THE DRAWINGS

FIG. 1-A and FIG. 1-B show a typical prior art stent of the BE type before and after deployment. Stent 1 is mounted on balloon 2 and inserted into a body vessel. After expansion the length of the stent is slightly shortened or stays the same but the diameter greatly increases, typically by a factor of 3 to 5 times. The balloon is guided by a catheter, which in turn can be guided by a guide wire (not shown). The technology of stent design is well known in the art and can be found in many scientific publications such as: “An Overview of Superelastic Stent Design” (Min Invas Ther & Allied Technology 2000: 9(3/4) pp 235-246) which is hereby incorporated by reference.

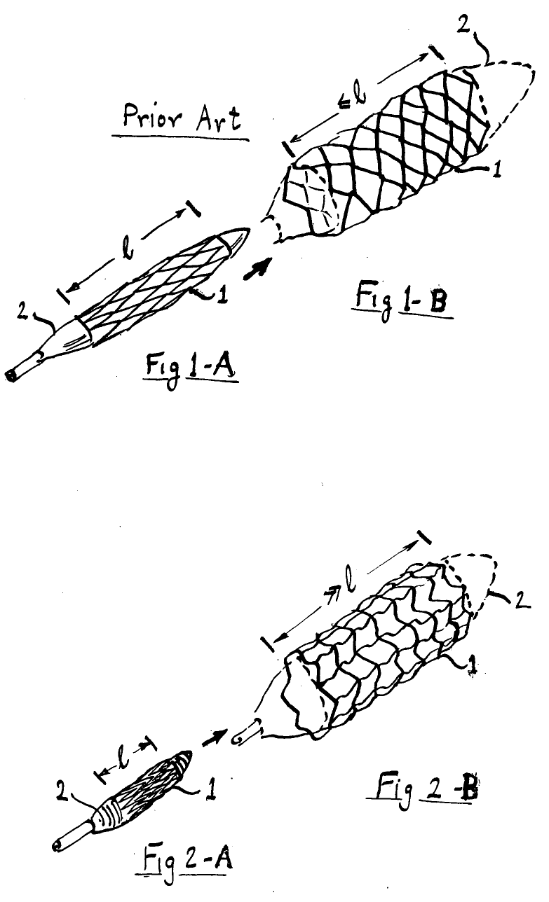

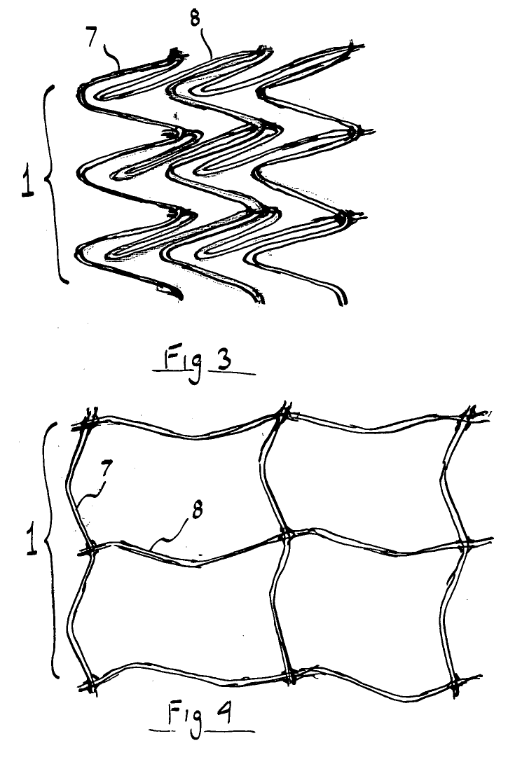

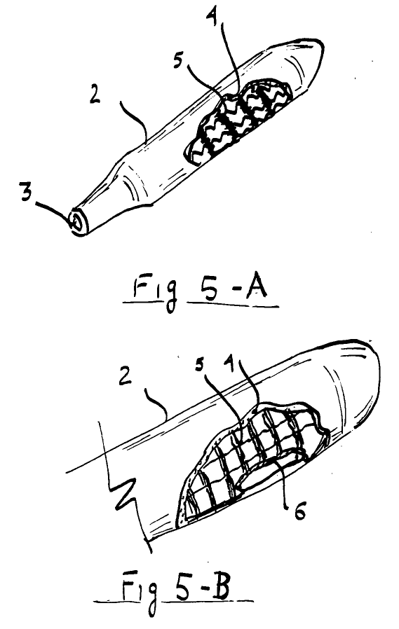

The Stent 1 according to the invention is shown in FIG. 2-A before deployment and in FIG. 2-B after deployment. The balloon 2 is shown in FIG. 2-B as a dotted line as it is removed (by deflating it) after deployment. The stent 1 according to the invention has a structure allowing significant elongation without loss of radial expansion. The preferred embodiment of such a structure is shown, greatly magnified, in FIG. 3. The stent comprises of a radially expandable structure 7, in the form of circumferentially expandable shapes, connected by axially expandable folded links 8. Typically stent 1 is machined (using laser cutting or other fine cutting method) from a piece of tubing. The material is any of the current material used for stents: type 316LVM stainless steel, Nitinol, titanium or any other bio-compatible material. Stents can be coated with special coatings including controlled drug elution and coatings for increased radio-opacity. Clearly the structure shown in FIG. 3 is shown by the way of example, and many structures can be designed, both BE and SE, which are capable of significant axial and radial expansion. FIG. 4 shows the invention in the expanded form, greatly magnified. The reason for not fully stretching the links 7 and 8 to be fully straight, is in order to maintain flexibility, both radially and axially. This flexibility is needed in order to allow the stent to flex with the vessel it is installed in. The greater the required stent flexibility the more links 7 and 8 need to remain flexible, usually achieved by using “S” shaped patterns instead of straight lines. In order to achieve maximum axial expansion, circumferential shapes 7 are designed to nest in each other as shown in FIG. 3. They can form a continuous spiral or separate rings connected by links 8. A special balloon, also capable of radial and axial expansion, is required for deployment of such BE stents and is shown in FIG. 5-A (before expansion) and FIG. 5 B (expanded). While any elastomeric balloon will expand both axially and radially when pressurized, the expansion will not be controlled. In the preferred embodiment the balloon is made of an elastomer such as silicone rubber re-enforced by filaments of a non-stretchable material such as Kevlar fiber. The fibers 4 are laminated between two elastomer layers, 4 and 6. The inflation is via opening 3. When a guide wire is used, a separate passage for the guide wire is provided (not shown) following prior art practice in catheter mounted balloons. Such catheter mounted balloons are commercially available and well known in the art. In the non-inflated form shown in FIG. 5-A the re-enforcing fibers are folded, similar to the folding of the links in the stent, allowing two-dimensional expansion. As soon as the fibers are fully stretched, very little further expansion occurs, as the balloon is no longer elastic. It is desirable to make the balloon more compliant in the axial direction than in the radial direction, as it is desired to expand the stent axially before it is fully expanded radially, in order to minimize rubbing against the vessel. This can be done by the use of an asymmetric mesh structure 5, having more re-enforcement in the circumferential direction. Because of the extra axial expansion higher pressures than used by standard balloons are needed. By the way of example, pressures in the range of 10-50 atmospheres are needed versus 5-20 atmospheres used in many current balloons. This assumes that stent dimensions and thickness of links are similar to prior art stents. Typical stents used today expand from 1-3 mm in diameter to 5-15 mm and their length is typically 10-50 mm, with about 0-10% length reduction during deployment. Typical cross section dimensions of the links are from 0.1 to 0.3 mm. Stents made according to the invention have similar properties to prior art stents except their length before expansion is reduced by a factor of 2 to 20 fold. According to the invention a BE stent could expand radially the same amount as a prior art stent while expanding axially by a factor of 2 to 20. The higher expansion ratios require thinner links, so a trade-off has to be made between expansion ratio, stiffness and flexibility in the expanded state.

For very large axial expansion ratios when using BE stents the preferred embodiment is shown in FIG. 9. Stent 1 is mounted on balloon 2, shown is the deflated state. Balloon 2 is guided by guide wire 13 and pressurized by tube 3 using liquid, typically a saline solution. Balloon 2 is made of silicone rubber or similar material and can be re-enforced with fibers as previously discussed. For disposable use other materials can be used, as balloon needs only to be used once. A bellows-like structure 16 allows very large axial expansion and also makes axial compliance lower than radial compliance. This is desired as it is preferred to expand the stent axially before it is fully expanded radially.

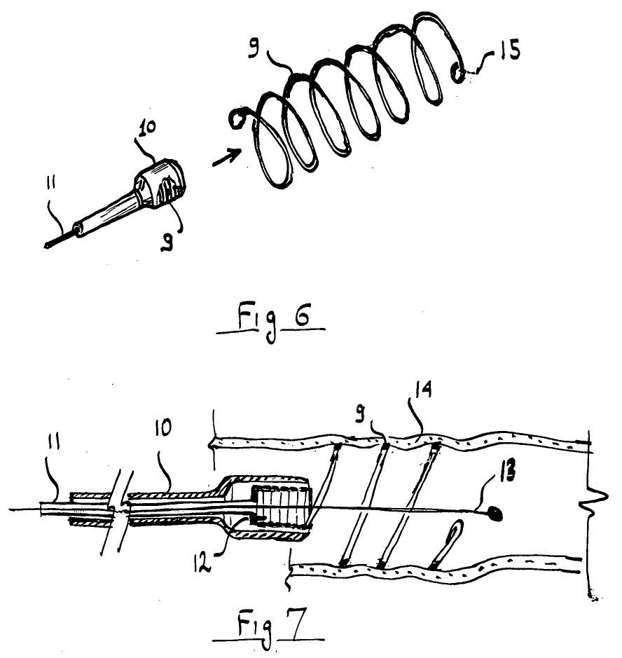

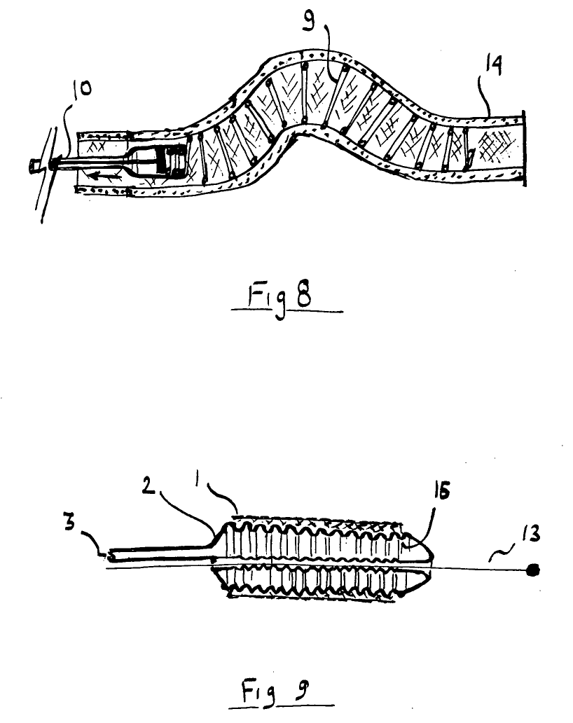

The invention can also be applied to SE stents, including those not deployed by balloons. The preferred embodiment of the invention in a SE stent is shown in FIG. 6. A spring 9, made from a ribbon of elastic or super-elastic alloy such as Nitinol, is coiled up and compressed into a hollow cylindrical tool 10. The spring can be pushed out from tool 10 by a push-wire 11. As soon as pushed out it expands both radially and axially to conform to the vessel, as shown in FIG. 7. The ends of the spring can be shaped like a closed loop 15. This not only avoids piercing the vessel wall, but allows removal of the stent, if required, by gripping loop 15 with a catheter equipped with a hook and winding spring 9 onto the catheter or pulling spring with the catheter. Such winding, pulling or a combination of both reduced the diameter sufficiently to pull stent out.

It is desired to make cross section of ribbon used for spring 9 rectangular. This way, the stiffness can be optimized separately for the axial and radial direction. Since stiffness increases with the third power of the dimension (ribbon width or thickness), large changes in stiffness can be achieved by small changes in dimensions. In FIG. 7 the deployment tool 10 is guided by a wire 13. The push wire 11 is made hollow to ride on guide wire 13, but can also easily be positioned besides it. The advantage of a concentric push wire 11 is that it can also be turned, slightly reducing diameter of coiled spring 9 inside tool 10 and lowering the force needed to push spring out. In such a case end 12 of wire 11 needs to have a pin to engage loop 15 at end of coil 9. While some of the examples given in the description relate to stents installed in blood vessels it is clear that the invention can be used in any body lumen such as esophogeal stents, colonic stents, urethral stents and many others. It is also clear that stents can be made of many different materials, not only metals. The art of stents is well known and the invention can be used in conjunction with other improvements in the art.

What is claimed is:

1. A stent capable of a significant increase in the axial dimension during deployment.

2. A stent as in claim 1 wherein the axial dimension is increased at least by 50% during deployment.

3. A balloon for deployment of stents as in claim 1, said balloon capable of both axial and radial expansion when pressurized.

4. A stent as in claim 1, wherein said stent is self-expanding.

5. A stent as in claim 1 wherein said stent is made in the form of a wound ribbon.

6. A stent as in claim 1 wherein said stent is removable.

7. A stent as in claim 1 wherein said stent has a functional coating.

8. A method of deploying a stent, said method comprising the steps of:
mounting said stent on a balloon capable of axial and radial expansion when pressurized;
inserting said balloon into a vessel inside the human body and pressurizing balloon; and
expanding said stent both radially and axially before removing pressure from said balloon.

9. A method of deploying a self expanding spring-like stent, said method comprising the steps of:
compressing said stent into a cylindrical cavity;
placing said cavity in a vessel inside the human body; and
releasing said stent from said cavity, allowing it to expand both radially and axially.
